(19) United States
(12) Patent Application Publication
(10) Pub. No.: US 2007/0232905 A1
(43) Pub. Date: Oct. 4, 2007

(54) Unconstrained Balloon Sizer

(76) Inventor: Tom J. Francis, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/278,592

(22) Filed: Apr. 4, 2006

(51) Int. Cl. A61B 5/107

(52) U.S. Cl. 600/432; 606/102; 600/594

(57) ABSTRACT

The embodiments provide systems and methods for determining at least one parameter of an intervertebral space. The systems and methods may be useful to determine the appropriate size and geometry of a spinal implant. Thus, the systems and methods provided by the embodiments may aid in the selection and implantation of a spinal implant, and may increase the probability of a positive surgical outcome.

### FIELD OF THE INVENTION

Embodiments relate to methods and systems for characterizing the intervertebral disc space. More particularly, embodiments relate to methods and systems for measuring the volume, dimensions, geometry, and other parameters of the intervertebral disc space using expandable members.

### BACKGROUND OF THE INVENTION

The intervertebral disc functions to stabilize the spine and to distribute forces between vertebral bodies. The natural intervertebral disc typically includes three structures: the nucleus pulposus, the annulus fibrosis, and two vertebral end-plates. The nucleus pulposus is an amorphous hydrogel in the center of the intervertebral disc. The annulus fibrosis, which is comprised mostly of highly structured collagen fibers, maintains the nucleus pulposus within the center of the intervertebral disc. The vertebral end-plates, primarily comprised of hyalin cartilage, separate the disc from adjacent vertebral bodies and act as a transition zone between the hard vertebral bodies and the soft disc.

Intervertebral discs may be displaced or damaged due to trauma, disease, or the normal aging process. One way to treat a displaced or damaged intervertebral disc is by surgical removal of a portion or all of the intervertebral disc, including the nucleus and the annulus fibrosis. However, the removal of the damaged or unhealthy disc may allow the disc space to collapse, which might in turn result in instability of the spine, abnormal joint mechanics, nerve damage, and severe pain. Therefore, after removal of the disc, a spinal implant such as a prosthetic nucleus, artificial disc, or fusion cage may be implanted in order to replace the removed nucleus or annulus, or a portion thereof.

Because the spinal implant replaces all or part of the intervertebral disc, it may be desirable to size the spinal implant according to the natural dimensions and geometry of the intervertebral disc that is to be replaced or augmented.

The description herein of problems and disadvantages of known devices and methods is not intended to limit the embodiments to the exclusion of these known entities. Indeed, embodiments may include one or more of the known devices and methods without suffering from the disadvantages and problems noted herein.

### SUMMARY

There is a need for systems and methods for determining various parameters of the intervertebral disc space such as the volume, dimensions, and geometry of the disc space or a portion thereof. The embodiments solve some or all of these needs, as well as additional needs.

Therefore, in accordance with an embodiment, there is provided a method for determining at least one parameter of an intervertebral disc space or an evacuated portion thereof. The method may comprise providing an expandable member having an internal cavity, the member being in fluid communication with a distal end of a longitudinal element having an axially concentric bore, and a stylet positioned within the longitudinal element's bore. The method also may comprise inserting the expandable member into the disc space, inflating the expandable member with a fluid until the expandable member has substantially occupied the disc space or an evacuated portion thereof, and measuring the volume of fluid in the expandable member.

In another embodiment, there is provided a method for imaging an intervertebral disc space or an evacuated portion thereof. The method may comprise providing an expandable member having an internal cavity, the member being in fluid communication with a distal end of a longitudinal element having an axially concentric bore, and a stylet positioned within the longitudinal element's bore. The method also may comprise inserting the expandable member into the disc space, inflating the expandable member with a fluid until the expandable member has substantially occupied the disc space or an evacuated portion thereof, and imaging the disc space and the expandable member while the expandable member is inflated in the disc space.

In another embodiment, there is provided a method for selecting an intervertebral disc device for implantation into an intervertebral disc space or an evacuated portion thereof. The method may comprise providing an expandable member having an internal cavity, the member being in fluid communication with a distal end of a longitudinal element having an axially concentric bore, and a stylet positioned within the longitudinal element's bore. The method also may comprise inserting the expandable member into the disc space, inflating the expandable member with a fluid until the expandable member has substantially occupied the disc space or an evacuated portion thereof, and determining at least one parameter of the disc space or an evacuated portion thereof. The intervertebral disc device may be selected at least in part on the basis of the determined parameter of the disc space.

In another embodiment, there is provided a device for determining at least one parameter of an intervertebral disc space. The device may comprise a longitudinal element having distal and proximate ends and an axially concentric bore, an expandable member comprising an internal cavity connected to and in fluid communication with the distal end of the longitudinal element, and a stylet capable of being inserted into the proximate end of the longitudinal element and capable of maintaining the expandable member in a substantially straight configuration during insertion of the expandable member into the disc space. The stylet may be substantially more flexible than the longitudinal element.

These and other features and advantages of the embodiments will be apparent from the description provide herein.

### BRIEF DESCRIPTION OF THE DRAWINGS

FIG. 1 is a drawing of an exemplary device according to the embodiments.

FIG. 2 is a drawing of an exemplary device according to the embodiments.

FIG. 3 is a drawing of an exemplary device according to the embodiments.

FIG. 4 is a drawing of an exemplary device according to the embodiments placed in an intervertebral disc space.

### DETAILED DESCRIPTION OF THE EMBODIMENTS

The following description is intended to convey a thorough understanding of the various embodiments by providing a number of specific embodiments and details involving methods and systems for determining at least one parameter of the intervertebral disc space, in particular for measuring the volume, dimensions, and geometry of the intervertebral disc space. It is understood, however, that the embodiments are not limited to these specifically preferred embodiments and details, which are exemplary only. It is further understood that one possessing ordinary skill in the art, in light of known systems and methods, would appreciate the use of the embodiments for their intended purposes and benefits in any number of alternative embodiments.

As used throughout this disclosure, the singular forms “a,” “an,” and “the” include plural reference unless the context clearly dictates otherwise.

Throughout this description, the expression “intervertebral disc space” may refer to any volume between two adjacent vertebrae. The intervertebral disc space may be the volume inside of the annulus fibrosis of the intervertebral disc. Alternatively, the intervertebral disc space also may include the annulus fibrosis itself.

What is meant by “primary axis,” as used in the specification, is the most important or longest axis of a component. For example, the “primary axis” of a longitudinal element that is bent somewhere along its length is the axis of the longest segment of the longitudinal element, the segments being defined by the bend in the element.

It is a feature of the embodiments to provide a device for determining at least one parameter of an intervertebral disc space. The device may comprise a longitudinal element having distal and proximate ends and an axially concentric bore, an expandable member comprising an internal cavity connected to and in fluid communication with the distal end of the longitudinal element, and a stylet capable of being inserted into the proximate end of the longitudinal element and capable of maintaining the expandable member in a substantially straight configuration during insertion of the expandable member into the disc space. The stylet preferably is substantially more flexible than the longitudinal element.

The longitudinal element may be used to deliver a fluid to the internal cavity of the expandable member and to insert the expandable member into an intervertebral disc space. The longitudinal element may have an optimal stiffness and flexibility to facilitate insertion into and maneuverability within the body. In a preferred embodiment, the distal end of the longitudinal element may be curved, bent, or angled relative to its primary axis. Alternatively, the longitudinal element may be easily deformable in order to conform to the intervertebral disc space. Additionally, the longitudinal element may have an optimal diameter for insertion into the body and delivery of the expandable member to the intervertebral disc space. It may be preferable that the diameter of the longitudinal element be not more than the height of the disc space and more preferably small enough to be inserted into the disc space using minimally invasive surgical techniques. For example, the longitudinal element may be no more than about 5 mm in diameter. One who is skilled in the art will appreciate how to choose the appropriate size and flexibility of the longitudinal element in accordance with the embodiments described herein.

The longitudinal element preferably comprises a connection mechanism at its proximate end in order to connect the longitudinal element to other equipment, such as the stylet provided by the embodiments, a fluid dispensing device, and so forth. Preferably, the proximate end of the longitudinal element has a male or female luer lock connector. Alternatively, the longitudinal element may have a luer slip connector or some other connection mechanism.

The expandable member may be connected to (e.g, by use of an adhesive) and in fluid communication with the distal end of the longitudinal element. Preferably, the expandable member is angled, curved, bent, or shifted relative to the primary axis of the longitudinal element. The expandable member may be any appropriate, biocompatible member having an internal cavity. Because the expandable member preferably is inserted into the body only for a momentary period of time, the expandable member need not be as biocompatible as a permanent implant. However, it is preferable that the expandable member be sufficiently biocompatible as to not cause any undesirable interactions during its brief insertion into the body. The expandable member preferably may be selected to withstand the pressure of inflation when a fluid is delivered to the expandable member so as to avoid rupture when inflated. Thus, the expandable member preferably has a mean burst pressure of at least about 22.4 psi.

In a preferred embodiment, the expandable member is a balloon. The balloon may be made of various polymeric materials such as polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof. ChronoPrene™, which is a blended polymer based on styrenic olefinic rubber and hydrogenated isoprene with polypropylene as a reinforcing agent and mineral oil as a plasticizer, and is commercially available from CardioTech International, Wilmington, Mass., is a preferred material for fabricating the expandable member.

Because the expandable member may be filled with image contrast agents and/or radioactive materials, it is preferred to fabricate the member from chemical-resistant materials. In addition, the expandable member may be made from a multi-layered material with an inner expandable chemically resistant layer, and/or the interior of the balloon may be coated with a chemically resistant coating.

The expandable member preferably is capable of withstanding a tensile stress of 8.9 Newtons without tearing, rupturing, or separating from the longitudinal element. Also, a tensile stress of 8.9 Newtons preferably stretches the expandable member a distance of at least about 50 mm without tearing. Because it is desirable that the expandable member be readily inflatable (i.e. capable of being inflated using little force), it may be preferable that the expandable member be capable of stretching relatively large distances under relatively little tensile stress. Thus, it may be preferable that the expandable member be capable of stretching about 40 mm, more preferably about 50 mm, even more preferably about 60 mm, and most preferably at least about 70 mm under a 9 Newton tensile stress. The expandable member also preferably is capable of withstanding 100 mL of inflation in an unconfined state (e.g. outside of the intervertebral disc space) without bursting.

In a more preferred embodiment, the expandable member is an unconstrained balloon. An unconstrained balloon is generally spherical or cylindrically shaped, and expands approximately equally in all directions when inflated so lono as it is not constrained by surrounding tissues (e.g. the interior surfaces of an intervertebral disc space). By comparison, the expansion of a constrained balloon may be limited by its inherent properties and/or materials so that it expands preferentially or selectively in certain directions when inflated. A constrained balloon, for example, may be shaped like a kidney or flattened disk when inflated. The preferred unconstrained balloon of the embodiments may be desirable because it can conform to volumes of varying geometry, whereas the constrained balloon conforms best to volumes shaped similar to the shape of the constrained balloon. Therefore, an unconstrained balloon may be more adaptable to various intervertebral disc space volumes than is a constrained balloon.

A stylet may be used to maintain the expandable member in a substantially straight configuration during insertion of the member into a disc space. The stylet may be an elongated rod with distal and proximate ends that is capable of being inserted or slipped into the axially concentric bore of the longitudinal element. The proximate end of the stylet preferably has a connection mechanism for engaging a complementary connection mechanism at the proximate end of the longitudinal element in order to hold the stylet in place inside of the longitudinal element. For example, if the proximate end of the longitudinal element has a male luer lock connector, the proximate end of the stylet preferably has a female luer lock connector, and vice-versa. Preferably, the distal end of the stylet is capable of extending beyond the distal end of the longitudinal element and into the expandable member. More preferably, the distal end of the stylet is capable of extending to approximately the distal end of the expandable member.

The stylet preferably straightens and/or maintains the expandable member in a substantially straight configuration during insertion of the expandable member into an intervertebral disc space. Without the stylet, the expandable member might be deformed, stretched, or snag on adjacent body members during insertion into the disc space. Thus, the stylet may facilitate or ease insertion of the expandable member into the disc space. In a preferred embodiment, the stylet extends slightly beyond the distal end of the expandable member when the expandable member is in a resting state, so that the stylet stretches the expandable member preferably into a shape having a reduced cross-sectional area. For example, if the expandable member is generally spherical in shape, the stylet may stretch the expandable member into a more cylindrical shape having a reduced cross-section.

Because the stylet preferably is intended only to straighten the expandable member, and not significantly stiffen the longitudinal element, it may be preferred that the stylet be substantially more flexible than the longitudinal element. Concurrently, it may be preferred that the stylet be substantially less flexible than the expandable member. In this manner, the stylet may be able to straighten the expandable member without adversely stiffening the longitudinal element. Additionally, the stylet may be bent or angled, for example, at its distal end in order to conform to a bent or angled longitudinal element. Preferably, both the stylet and the longitudinal element bend or angle can be adjusted by applying pressure to the stylet and/or longitudinal element.

A fluid dispensing device may be connected to the proximate end of the longitudinal element. The dispensing device may be capable of holding a fluid and adapted to be connected to and in fluid communication with the proximate end of the longitudinal element. Therefore, the dispensing device may be used to deliver a fluid to the longitudinal element and expandable member at the element's distal end, thus inflating the expandable member. Preferably, the dispensing device is a syringe. More preferably, the dispensing device is a syringe graduated by volume. By noting the volume of fluid in the syringe before and after inflation of the expandable member, one of ordinary skill in the art may be able to compute the inflation volume of the expandable member.

The dispensing device preferably has a connection mechanism for engaging a complementary connection mechanism at the proximate end of the longitudinal element. For example, if the proximate end of the longitudinal element has a male luer lock connector, the dispensing device preferably has a female luer lock connector, and vice-versa. Alternatively, the proximate end of the longitudinal element may include a seal that can be repeatedly punctured by a needle on the dispensing device, much like a medicine vial. Other connection devices including, but not limited to, luer slip connectors, also may be used to detachably connect the dispensing device to the proximate end of the longitudinal element. The dispensing device may be used to draw a fluid from a separate container and then deliver the fluid to the longitudinal element.

The fluid may be selected from, for example, saline solutions and imaging contrast mediums. Imaging contrast mediums, of course, are preferred where it is desired to also image the disc space while the expandable member is inflated therein.

The imaging contrast media contemplated for use in the embodiments include all applicable imaging contrast media, including contrast agents for X-ray, derivative (commercially available from Mallinckrodt Imaging, Tyco Healthcare, Mansfield, Mass.), iohexol (Omnipaque®, commercially available from GE Healthcare, Chalfont St. Giles, United Kingdom), iopamidol (Isovue®, commercially available from Bracco Diagnostics, Princeton, N.J.), ioversol (Optiray®, commercially available from Mallinckrodt Imaging, Tyco Healthcare, Mansfield, Mass.), and iopromide (Ultravist®, commercially available from Berlex Imaging, Montville, N.J.).

Specific MRI imaging contrast media contemplated for use in the embodiments include, but are not limited to, gadolinium derivatives and complexes such as gadoteridol, gadoterate meglumine, gadodiamide, and gadopentetate (Magnevist®, commercially available from Berlex Imaging, Montville, N.J.); iron derivatives and complexes; manganese derivatives and complexes such as mangafodipir trisodium; superparamagnetic iron oxide contrast medias; ferumoxides such as FERIDEX® (commercially available from Berlex Imaging, Montville, N.J.); and perfluorocarbons. The MRI imaging contrast media may be either positive or negative contrast media.

It may be desirable that the MRI imaging contrast media comprise complexes of a complexing agent and a metal such as gadolinium, manganese, or iron. Exemplary complexing agents include, but are not limited to, diethylenetriamne-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); X-ray technologies such as CT (computerized tomography) and C-arm fluoroscopy (e.g. Iso-C technology available from Siemens AG, Berlin, Germany), MRI (magnetic resonance imaging), and PET (positron emission tomography) imaging. Typically, the imaging contrast medium may be chosen to correspond to the imaging technique to be used. For example, if X-ray images are to be taken of the inflated expandable member, then X-ray imaging contrast media preferably may be used. Similarly, if images are to obtained using an MRI technique, then MRI imaging contrast media preferably may be used. Additionally, it may be preferable that the imaging contrast medium comprise a fluid or liquid solution, gel, paste, or suspension of an X-ray, CT, MRI, and PET contrast agent rather than an aqueous composition containing the contrast agent. Therefore, it should be understood that imaging contrast media may comprise fluid or liquid solutions, gels, pastes, and suspensions of X-ray, CT, MRI, and PET contrast agents in addition to the contrast agent itself, One who is skilled in the art will appreciate the wide array of imaging contrast media that may be used in accordance with the embodiments.

Specific X-ray imaging contrast media contemplated for use in the embodiments include, but are not limited to, barium sulfate, acetrizoic acid derivatives, diatrizoic acid derivatives such as Hypaque® (commercially available from Amersham, GE Healthcare, Chalfont St. Giles, United Kingdom), diatrizoate meglumine/sodium, iothalamic acid derivatives, iothalamates, ioxithalamic acid derivatives, iothalamate meglumine, metrizoic acid derivatives, iodide, iodipamide meglumine, ioglycamic acid, dimeric ionic contrast agents, ioxaglic acid derivatives, metrizamide, metrizoate, iopamidol, iohexol, iopromide, iobitridol, iomeprol, iopentol, ioversol, ioxilan, iodixanol, iotrolan, ioxaglate (Hexabrix®, commercially available from Mallinckrodt Imaging, Tyco Healthcare, Mansfield, Mass.), ioxaglate meglumine/sodium, iotrol, iopanoic acid, and organic radiographic iodinated contrast media (ICM) such as modifications of a 2,4,6-tri-iodinated benzene ring including Renografin® (commercially available from Amersham, GE Healthcare, Chalfont St. Giles, United Kingdom), Conray® trans(1,2)-cyclohexane diethylene triamine pentaacetic acid (“CDTPA”); 1-oxa-4,7,10-triazacyclododecane-N,N′N″-triacetic acid (“OTTA”); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives and analogs thereof, particularly protected forms of the compounds.

CT scan imaging contrast media contemplated for use in the embodiments include orally, intravenously, and rectally administered media. Specific CT scan imaging contrast media contemplated for use in the embodiments include, but are not limited to, iodine solutions, barium sulfate, mixtures of sodium amidotrizoate and meglumine amidotrizoate (such as Gastrografin®, commercially available from Bristol-Myers Squibb, Princeton, N.J.), and, in general, the imaging contrast media mentioned previously in relation to X-rays.

PET scan imaging contrast media typically comprise a positron emitting (i.e. radioactive) element incorporated into a carrier such as a complexing agent or a biologically active molecule such as glucose. Specific PET scan imaging contrast media contemplated for use in the embodiments include, but are not limited to, complexes and derivatives of positron emitting radioisotopes including, but not limited to, carbon-11, nitrogen-13, oxygen-15, fluorine-18, iron-52, cobalt-55, copper-62, copper-64, bromine-75, bromine-76, technetium-94m, gallium-68, gallium 66, sellenium-73, bromine-75, bromine-76, iodine-120, iodine-124, and indium-110m. These radioactive elements may be incorporated into a carrier such as an organic molecule that is fluid at room temperature. Alternatively, these radioisotopes may be complexed with a complexing agent such as the complexing agents previously mentioned in regards to MRI imaging contrast media and placed in solution. Because the PET imaging contrast media are to be used in the expandable members placed inside the body, it may be preferable to choose PET imaging contrast media with short half-lives to reduce the risk to the patient in the event of a rupture of the expandable member. For example, PET imaging contrast media with a half-life of about 2 hours such as gallium-68 are preferred. PET scans can be adopted to the embodiments simply by injecting an applicable PET imaging contrast medium into the expandable member, thereby rendering the expandable member easily detectible by the PET scanning instrument.

In another embodiment, the imaging contrast media may include a metallic radioisotope including, but not limited to, the isotopes actinium-225, astatine-211, iodine-120, iodine-123, iodine-124, iodine-125, iodine-126, iodine-131, iodine-133, bismuth-212, arsenic-72, bromine-75, bromine-76, bromnine-77, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium 186, rhenium-188, tellurium- 121m, tellurium-122m, tellurium-125m, thulium-165, thulium-167, thulium-168, technetium-94m, technetium-99m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, and ytterbium-169.

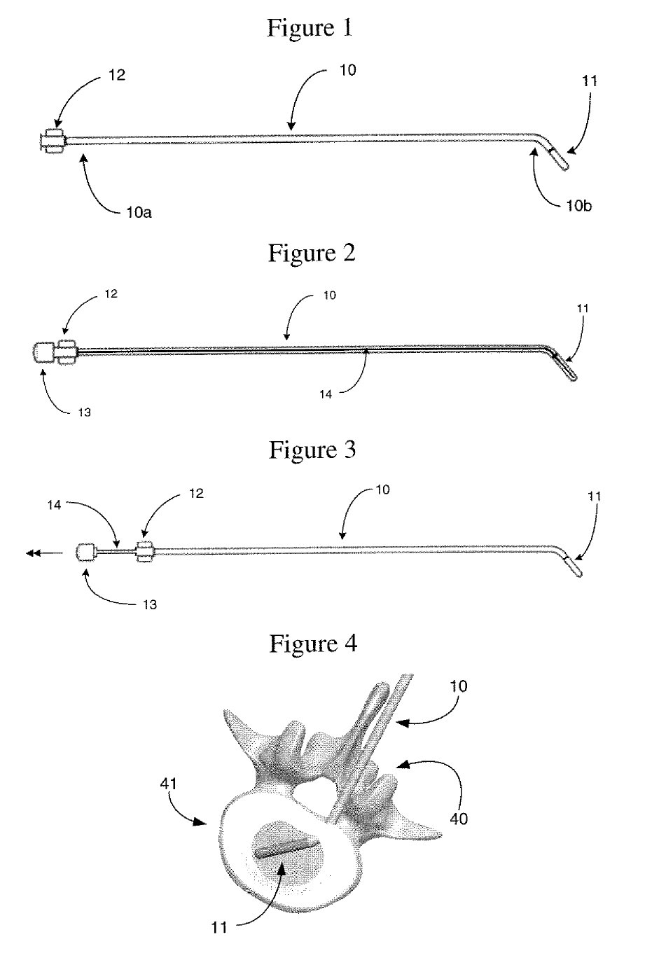

FIG. 1 illustrates an exemplary device according to the embodiments. A longitudinal element 10 is provided, having proximate 10a and distal 10b ends. As seen, the longitudinal element optionally may be bent, for example near its distal end 10b. This may be desirable in order to ease insertion of the longitudinal element and associated expandable member into a disc space. It will be appreciated that, despite the bend at its distal end, the primary axis of the longitudinal element 10 in FIG. 1 is substantially horizontal. An expandable member 11 is attached to the distal end of the longitudinal element. In this example, the expandable member 11 is a cylindrically shaped polymeric balloon with approximately the same cross-section as the longitudinal element. A balloon with the same cross-section as the longitudinal element may be desirable in order to ease insertion of the device into an intervertebral disc space. Preferably, as shown in FIG. 1, the expandable member is angled, bent, or shifted relative to the primary axis of the longitudinal element 10 in order to, for example, ease insertion of the expandable member into a disc space. An optional female luer lock connector 12 is attached to the proximate end of the longitudinal element.

FIG. 2 illustrates another exemplary device according to the embodiments. FIG. 2 shows a longitudinal element 10, an expandable member 11 attached to the distal end of the longitudinal element, and an optional female luer lock connector 12 attached to the proximate end of the longitudinal element. Also, a male luer lock connector 13 is provided in engagement with the female luer lock connector. The male luer lock connector is attached to the proximate end of a stylet 14 positioned inside the axial bore of the longitudinal element (the longitudinal element is depicted as being translucent so that the stylet 14 may be seen inside of it). It will be appreciated that the male and female luer lock connectors may be switched, or substituted with another connector such as a luer slip connector. In any case, it is desirable that some form of connection mechanism be provided in order to selectively connect the stylet with the longitudinal element in order to hold the stylet in place inside of the element during insertion of the expandable member into a disc space. Preferably, as shown, the stylet extends to the distal end of the expandable member.

FIG. 3 illustrates the stylet 14 being withdrawn from the longitudinal element 10 by retracting the stylet from the proximate end of the longitudinal element. Of course, if the proximate ends of the longitudinal element and the stylet have complementary connectors, then the connectors will have been disengaged prior to withdrawing or removing the stylet from the longitudinal element. Because the stylet may substantially block the axial bore in the longitudinal element, it may be necessary to remove the stylet before a fluid can be delivered to the expandable member through the longitudinal element. Alternatively, the stylet may be sized and/or shaped so that fluid is able to pass by the stylet and through the longitudinal element to reach the expandable member, so that the stylet need not be removed in order to inflate the expandable member.

FIG. 4 illustrates placement of the expandable member 11 of the device into an intervertebral disc space 41. As seen, the longitudinal element 10 facilitates placement of the expandable member 11 from beyond the vertebral body 40. Thus placed, the expandable member may be inflated to substantially occupy the disc space or an evacuated portion thereof. By observing the volume of fluid required to inflate the expandable member 11 until the disc space or an evacuated portion thereof is occupied, the volume of the disc space or evacuated portion thereof may be determined. Additionally, the inflated expandable member and disc space may be imaged in order to determine further parameters of the disc space.

In a preferred embodiment, a pressure measurement device may be connected to and in fluid communication with the longitudinal element. The pressure measurement device may be used to monitor the pressure of the fluid as it is delivered to the longitudinal element and the connected expandable member. In another preferred embodiment, a catheter, cannula, or trocar may be provided that is coaxial to the longitudinal element. The catheter, cannula, or trocar may function as a guide to facilitate insertion of the longitudinal element and the expandable member into the body. The catheter, cannula, or trocar preferably may sheath the longitudinal element and the expandable member. In another preferred embodiment, the device may additionally comprise a guidewire positioned within the longitudinal element. The guidewire may be used to guide the longitudinal element during insertion so as to more easily place the longitudinal element at the desired position in the body, for example immediately adjacent to or inside of the intervertebral disc space.

In another embodiment, there is provided a surgical kit. The surgical kit may comprise a longitudinal element having distal and proximate ends; an expandable member that is capable of being attached to the distal end of the longitudinal element; a dispensing device such as a syringe that is capable of being attached to the proximate end of the longitudinal element; and a stylet that is capable of being inserted into the longitudinal element. In a preferred embodiment, the kit may further comprise one or more of a pressure measurement device; a catheter, cannula, or trocar; and a guidewire. Each component of the kit may be connected to one or more other components or detached but capable of being connected to the other components.

The embodiments also provide a method for determining at least one parameter of an intervertebral disc space or an evacuated portion thereof. The method may comprise providing an expandable member having an internal cavity, the member being in fluid communication with a distal end of a longitudinal element having an axially concentric bore, and a stylet positioned within the longitudinal element's bore. The method also may comprise inserting the expandable member into the disc space, inflating the expandable member with a fluid until the expandable member has substantially occupied the disc space or an evacuated portion thereof, and measuring the volume of fluid in the expandable member.

In another embodiment, there is provided a method for imaging an intervertebral disc space or an evacuated portion thereof. The method may comprise providing an expandable member having an internal cavity, the member being in fluid communication with a distal end of a longitudinal element having an axially concentric bore, and a stylet positioned within the longitudinal element's bore. The method also may comprise inserting the expandable member into the disc space, inflating the expandable member with a fluid until the expandable member has substantially occupied the disc space or an evacuated portion thereof, and imaging the disc space and the expandable member while the expandable member is inflated in the disc space. This method can be used to determine whether the disc space has been evacuated by the surgeon to the desired degree of evacuation (e.g., partial or full).

In another embodiment, there is provided a method for selecting an intervertebral disc device for implantation into an intervertebral disc space or an evacuated portion thereof. The method may comprise providing an expandable member having an internal cavity, the member being in fluid communication with a distal end of a longitudinal element having an axially concentric bore, and a stylet positioned within the longitudinal element's bore. The method also may comprise inserting the expandable member into the disc space, inflating the expandable member with a fluid until the expandable member has substantially occupied the disc space or an evacuated portion thereof, and determining at least one parameter of the disc space or an evacuated portion thereof. The intervertebral disc device may be selected at least on the basis of the at least one determined parameter of the disc space.

The methods provided by the embodiments may be used for determining parameters of an intervertebral disc space such as the volume, dimensions, and geometry of an intervertebral disc space. In an exemplary embodiment, an expandable member is inserted into the intervertebral disc space. Preferably, the expandable member may be inserted using minimally invasive surgical techniques. For example, a longitudinal element such as described herein may be used to insert the expandable member into the intervertebral disc space. One who is skilled in the art will appreciate other ways in which to introduce an expandable member into an intervertebral disc space.

The intervertebral disc space may be partially or fully evacuated before insertion of the expandable member. Partial or full evacuation of the intervertebral disc space may be accomplished, for example, by removing at least a portion of the nucleus and/or annulus of the intervertebral disc space before insertion of the expandable member. For example, a degenerated or undesired portion of the nucleus and/or annulus of the intervertebral disc may be removed before insertion of the expandable member. Alternatively, a complete nucleotomy or discectomy may be performed to remove the nucleus or entire intervertebral disc before insertion of the expandable member. Methods of accessing the disc space, and removing a portion or all of the nucleus and/or annulus are well known in the art, and applicable for use in the embodiments disclosed herein.

During insertion of the member into the disc space, the stylet may be positioned inside of the longitudinal element and expandable member in order to substantially straighten the expandable member. Following insertion of the expandable member into the disc space, the stylet may be removed and a dispensing device may be attached to the longitudinal element. The expandable member then may be inflated with a fluid provided by the dispensing device. For example, a syringe graduated by volume as described in the embodiments herein may be used as the dispensing device, and may be attached to the longitudinal element's proximate end after the stylet is removed. The fluid used to inflate the expandable member preferably is a saline solution or an imaging contrast medium.

The expandable member preferably will inflate at a steady rate until the member has substantially occupied the disc space or an evacuated portion thereof, at which point the expandable member will become much more difficult to inflate. Preferably, inflation of the expandable member may be stopped at this point. Alternatively, the pressure of the expandable member may be monitored using a pressure measurement device attached to the longitudinal element, and inflation may be stopped when the pressure begins to rise more rapidly after a previously steady increase. In another alternative, inflation of the expandable member may be monitored using radio-fluoroscopy, and inflation may be stopped once it is observed that the expandable member has substantially occupied the disc space or an evacuated portion thereof.

The volume of fluid required to inflate the expandable member may be noted. One of ordinary skill in the art will be able to compute the volume of the inflated expandable member in part on this basis. For example, the volume of the inflated expandable member may be computed by subtracting the volume of the axially concentric bore of the longitudinal element by which the dispensing device is connected to the expandable member from the volume of the fluid dispensed by the dispensing device. Because the expandable member preferably is inflated until it substantially occupies an intervertebral disc space or an evacuated portion thereof, the methods of the embodiments provide a way of measuring the volume of the occupied intervertebral disc space or evacuated portion thereof.

In addition to or as an alternative to measuring the volume of the intervertebral disc space or an evacuated portion thereof, the inflated expandable member and the disc space may be imaged, for example using imaging procedures known to one of ordinary skill in the art. If imaging of the disc space is to be performed, the expandable member preferably will be inflated with an appropriate imaging contrast medium such as the X-ray, CT scan, MRI, and PET scan contrast media described herein. Alternatively, the expandable member may be coated with an imaging contrast medium before inserting the member into the disc space. In this way, the quality of the image obtained may be increased, compared to inflation using saline solution or some other non-radiographic fluid.

Parameters that can be measured by imaging the disc space and inflated expandable member in accordance with the embodiments include one-dimensional parameters such as the anterior-posterior width, lateral width, and height of the intervertebral disc space. Additionally, two-ditmensional parameters such as the cross-sectional areas of the intervertebral disc space perpendicular (i.e. “footprint”) and parallel (i.e. “projected”) to the spinal column can be determined. Simple imaging techniques such as X-ray may be useful to determine the cross-sectional area of the intervertebral disc space parallel to the spinal column, but more advanced imaging techniques such as CT, C-arm fluoroscopy, MRI, and PET technologies preferably are used to determine the cross-sectional area of the disc space perpendicular to the spinal column. Additionally, three-dimensional parameters of the intervertebral disc space such as the volume and geometry (e.g. topography) of the disc space may be determined.

Where a computerized imaging technique is used, parameters of the disc space may be determined by a computer analyzing the obtained images. For example, a computer may compute the volume of the intervertebral disc space or cross-sectional areas of the disc space on the basis of the obtained images. In both computational and non-computational imaging techniques, it may be advantageous to include a dimensional reference in the images in order to normalize the observed dimensions of the disc space. For example, a metal structure such as a rod of known dimensions may be placed adjacent to the intervertebral disc space (e.g. on the skin of the patient at a location adjacent to the disc space) prior to imaging such that the rod will appear in the images obtained of the disk space. In this manner, the length of dimensions observed in the images may be normalized to the known length of the dimensional reference.

One who is skilled in the art will appreciate the existing procedures and methods by which radiography may be carried out. The inflated expandable member may be imaged with any applicable imaging regime, technique, or technology. Preferred methods of imaging the inflated expandable member include X-ray, derivative X-ray technologies such as CT (computerized tomography) and C-arm fluoroscopy (e.g. Iso-C technology available from Siemens AG, Berlin, Germany), MRI, and PET scan. In a preferred embodiment, the imaging contrast medium may be selected to correspond to the method of imaging that is to be used. The inflated expandable member may be imaged once or a multiple of times. In another embodiment, more than one imaging method may be used. If more than one imaging method is to be used, it may be preferable to inflate the expandable member with an imaging contrast medium appropriate for one of the imaging methods, deflate the expandable member, and then inflate the expandable member again, but with a different imaging contrast medium appropriate for another imaging method. This may be repeated for each imaging method to be used.

The expandable member may be deflated and removed from the intervertebral disc space after determining the volume of the occupied disc space or portion thereof, or after determining some other parameter. Preferably, the expandable member may be removed from the intervertebral disc space in a minimally invasive manner, for example, using the longitudinal element to which the member is attached.

The systems and methods of the embodiments may be advantageously used to determine various intervertebral disc parameters such as the volume, dimensions, and geometry prior to implantation of a spinal implant. The parameters obtained by use of the expandable members may be used to select a spinal implant prior to implantation. Selection prior to implantation may be advantageous because of the reduced surgical time and increased likelihood of a desirable clinical result.

The spinal implant may be any implant used to replace all or part of the nucleus and/or annulus of the intervertebral disc, for example a fusion cage, artificial disc, or prosthetic disc nucleus. A snug fit between the spinal implant and the intervertebral disc space is thought to be desirable because of the reduced possibility of implant rotation, reduced possibility of excessive implant movement inside of the disc space, increased contact between the vertebral end plates and implant, and increased annulus tension. Therefore, a correctly sized spinal implant may be more likely to achieve a desirable clinical result than would be an incorrectly sized implant.

In another embodiment, excess tissue may be removed before implantation of the spinal implant. Because implants typically are manufactured pre-surgery, it may be easier to shape the intervertebral disc space to fit the implant than it is to shape the implant to conform to the intervertebral disc space. Imaging of the inflated expandable member and the determination of various parameters of the disc space such as the dimensions, volume, and geometry of the intervertebral disc space therefore may enable a surgeon to determine what, if any, excess discal tissue should be removed prior to implantation of the spinal implant. This may lead to a closer correlation in size and shape between the intervertebral disc space and the spinal implant, and a more desirable clinical outcome.

The foregoing detailed description is provided to describe the embodiments in detail, and is not intended to limit the embodiments. Those skilled in the art will appreciate that various modifications may be made to the embodiments without departing significantly from the spirit and scope thereof.

What is claimed is:

1. A method for determining at least one parameter of an intervertebral disc space or an evacuated portion thereof, comprising:
providing an expandable member having an internal cavity, the member being in fluid communication with a distal end of a longitudinal element having an axially concentric bore, and a stylet positioned within the longitudinal element's bore;
inserting the expandable member into the disc space;
inflating the expandable member with a fluid until the expandable member has substantially occupied the disc space or an evacuated portion thereof; and
measuring the volume of fluid in the expandable member.

2. The method of claim 1, further comprising at least partially evacuating a disc space prior to inserting the expandable member.

3. The method of claim 1, further comprising withdrawing the stylet from the longitudinal element after inserting the expandable member into the disc space and before inflating the expandable member.

4. The method of claim 1, wherein the stylet is capable of maintaining the expandable member in a substantially straight configuration during insertion of the expandable member into the disc space.

5. The method of claim 1, further comprising providing a syringe graduated by volume and capable of attaching to the longitudinal element's proximate end.

6. The method of claim 5, wherein inflating the expandable member comprises attaching the graduated syringe to the longitudinal element's proximate end and injecting a fluid from the syringe into the longitudinal element and expandable member.

7. The method of claim 1, wherein the fluid is selected from saline solution and an imaging contrast medium.

8. The method of claim 1, further comprising imaging the disc space and the expandable member while the expandable member is inflated in the disc space.

9. The method of claim 8, wherein the fluid is an imaging contrast medium.

10. The method of claim 8, further comprising coating the expandable member with an imaging contrast medium prior to inserting the expandable member into the disc space.

11. The method of claim 1, wherein the expandable member is a chronoprene balloon.

12. A method for imaging an intervertebral disc space or an evacuated portion thereof, comprising:
providing an expandable member having an internal cavity, the member being in fluid communication with a distal end of a longitudinal element having an axially concentric bore, and a stylet positioned within the longitudinal element's bore;
inserting the expandable member into the disc space;
inflating the expandable member with a fluid until the expandable member has substantially occupied the disc space or an evacuated portion thereof; and
imaging the disc space and the expandable member while the expandable member is inflated in the disc space.

13. The method of claim. 12, further comprising at least partially evacuating a disc space prior to inserting the expandable member.

14. The method of claim 12, further comprising withdrawing the stylet from the longitudinal element after inserting the expandable member into the disc space and before inflating the expandable member.

15. The method of claim 12, wherein the stylet is capable of maintaining the expandable member in a substantially straight configuration during insertion of the expandable member into the disc space.

16. The method of claim 12, further comprising measuring the volume of fluid in the expandable member when it has been inflated to substantially occupy the disc space or an evacuated portion thereof.

17. The method of claim 12, further comprising providing a syringe graduated by volume and capable of attaching to the longitudinal element's proximate end.

18. The method of claim 17, wherein inflating the expandable member comprises attaching the graduated syringe to the longitudinal element's proximate end and injecting a fluid from the syringe into the longitudinal element and expandable member.

19. The method of claim 12, wherein the fluid is selected from saline solution and an imaging contrast medium.

20. The method of claim 12, further comprising coating the expandable member with an imaging contrast medium prior to inserting the expandable member into the disc space.

21. The method of claim 12, wherein the expandable member is a chronoprene balloon.

22. A method for selecting an intervertebral disc device for implantation into an intervertebral disc space or an evacuated portion thereof, comprising: providing an expandable member having an internal cavity, the member being in fluid communication with a distal end of a longitudinal element having an axially concentric bore, and a stylet positioned within the longitudinal element's bore;
inserting the expandable member into the disc space;
inflating the expandable member with a fluid until the expandable member has substantially occupied the disc space or an evacuated portion thereof; and
determining at least one parameter of the disc space or an evacuated portion thereof;
wherein the device is selected at least on the basis of the determined parameter of the disc space.

23. The method of claim 22, wherein determining at least one parameter of the disc space or an evacuated portion thereof comprises the volume of fluid in the expandable member when the member has been inflated to substantially occupy the disc space or an evacuated portion thereof.

24. The method of claim 22, wherein determining at least one parameter of the disc space or an evacuated portion thereof comprises imaging the disc space and the expandable member while the expandable member is inflated therein.

25. The method of claim 24, wherein the fluid is an imaging contrast medium.

26. The method of claim 24, further comprising coating the expandable member with an imaging contrast medium prior to inserting the expandable member into the disc space.

27. The method of claim 22, further comprising withdrawing the stylet from the longitudinal element after inserting the expandable member into the disc space and before inflating the expandable member.

28. The method of claim 22, wherein the stylet is capable of maintaining the expandable member in a substantially straight configuration during insertion of the expandable member into the disc space.

29. The method of claim 22, further comprising providing a syringe graduated by volume and capable of attaching to the longitudinal element's proximate end.

30. The method of claim 29, wherein inflating the expandable member comprises attaching the graduated syringe to the longitudinal element's proximate end and injecting a fluid from the syringe into the longitudinal element and expandable member.

31. A device for determining at least one parameter of an intervertebral disc space, comprising:
a longitudinal element having distal and proximate ends and an axially concentric bore;
an expandable member comprising an internal cavity connected to and in fluid communication with the distal end of the longitudinal element; and
a stylet capable of being inserted into the proximate end of the longitudinal element and capable of maintaining the expandable member in a substantially straight configuration during insertion of the expandable member into the disc space;
wherein the stylet is substantially more flexible than the longitudinal element.

32. The device of claim 31, wherein the stylet extends approximately to a distal end of the expandable member.

33. The device of claim 31, wherein a proximate end of the expandable member is attached by an adhesive to the distal end of the longitudinal element.

34. The device of claim 31, further comprising a male or female luer lock connector at the proximate end of the longitudinal element.

35. The device of claim 31, further comprising a male or female luer lock connector at the proximate end of the stylet.

36. The device of claim 31, further comprising a dispensing device that is capable of holding a fluid and adapted to be connected to and in fluid communication with the proximate end of the longitudinal element.

37. The device of claim 36, wherein the dispensing device is a syringe.

38. The device of claim 37, wherein the syringe comprises a male or female luer lock connector at its distal end.

39. The device of claim 37, wherein the syringe is graduated by volume.

40. The device of claim 31, wherein the expandable member is angled relative to the primary axis of the longitudinal element.

41. The device of claim 31, wherein the longitudinal element is angled at its distal end relative to its primary axis.

42. The device of claim 31, wherein the stylet is angled at its distal end relative to its primary axis.

43. The device of claim 31, wherein the expandable member is a chronoprene balloon.

44. The device of claim 31, wherein a tensile stress of 8.9 Newtons stretches the expandable member a distance of at least about 50 mm without tearing.
